(19) United States
(12) Patent Application Publication
(10) Pub. No.: US 2023/0172509 A1
(43) Pub. Date: Jun. 8, 2023

(54) METHODS FOR TREATING SOCIAL DISORDERS

(71) Applicant: SAN DIEGO STATE UNIVERSITY RESEARCH FOUNDATION, San Diego, CA (US)

(72) Inventor: Nader Amir, San Diego, CA (US)

(21) Appl. No.: 18/105,111

(22) Filed: Feb. 2, 2023

Related U.S. Application Data
(63) Continuation-in-part of application No. 12/743,357, filed on Sep. 23, 2010, now abandoned.
(60) Provisional application No. 60/988,666, filed on Nov. 16, 2007.

(51) Int. Cl. A61B 5/16; G16H 20/30; G16H 70/20

(52) U.S. Cl. CPC A61B 5/165; G16H 20/30; G16H 70/20

(57) ABSTRACT

Systems and methods for treating patients with an anxiety disorder are disclosed. The systems comprise a screen for displaying sets of stimuli, a computer to control the display of stimuli onto the screen during at least one treatment session and the ability for the patient to interact with the screen in response to the displayed stimuli. The interaction of the patient with the system during the treatment session is capable of treating patient anxiety associated with an anxiety disorder, such as social anxiety. Also provided are computer programs capable of being used in the systems and methods of the present invention for treating anxiety.

### STATEMENT REGARDING FEDERALLY SPONSORED RESEARCH OR DEVELOPMENT

Part of the work performed during development of this invention utilized U.S. Government funds under NIH contracts R34MH077129 and R34MH07300401. The U.S. Government has certain rights in this invention.

### BACKGROUND OF THE INVENTION

### Field of the Invention

The present invention relates to systems and methods for treating patients with a social anxiety disorder. The systems comprise a screen for displaying sets of stimuli, a computer to control the display of stimuli onto the screen during at least one treatment session and a means for the patient to interact with the screen in response to the displayed stimuli. The interaction of the patient with the system during the treatment session is capable of treating patient anxiety associated with social settings. The invention also relates to computer programs capable of being used in the systems and methods of the present invention for treating anxiety.

### Background of the Invention

Anxiety is a physiological state characterized by cognitive, somatic, emotional, and behavioral components. Anxiety is manifested by feelings of fear, apprehension, or worry, and physiologically by heart palpitations, nausea, chest pain, shortness of breath, or headache. Although considered a normal behavior in humans, it can be characterized as a disorder in certain individuals. The four most common types of anxiety disorders are post-traumatic stress disorder (PTSD), generalized anxiety disorder, panic disorder, and social anxiety disorder.

Numerous psychological and biochemical factors are thought to contribute to anxiety disorders. Social anxiety, also known as social phobia, is sometimes thought to be related to an imbalance of the brain chemical serotonin. However, various psychological factors contribute to social anxiety, such as core negative beliefs based on personality and individual negative experiences. Anxiety disorders are still believed to be under-recognized in patients, and usually only diagnosed upon the onset of more severe complications such as depression or substance abuse.

Current treatments of anxiety disorders include medication or a short-term psychotherapy that is known as Cognitive-behavioral therapy. Medications are effective to treat some anxiety disorders, but are costly and often have unwanted side-effects. Typical Cognitive-behavioral therapy techniques, although believed to be effective, usually involve one-on-one direct interaction with a therapist, which requires significant expense and time. Thus, it is desired to develop methods for treating anxiety disorders that allow a patient to undergo treatment sessions that are convenient and easy for a patient and without the need for constant interaction with a therapist.

Cognitive theorists argue that attention processes play an important role in the maintenance of pathological anxiety (e.g., Beck, Emery, & Greenberg, 1985; Mogg & Bradley, 1998; Williams, Watts, MacLeod, & Mathews, 1997). A large body of empirical evidence suggests that anxious individuals selectively attend to threatening information (e.g., Mathews & MacLeod, 2005; Williams et al., 1997). Attentional processes are thought to be particularly important in that attention selectively facilitates the early processing of threat, thereby influencing subsequent cognitive, behavioral, and emotional processes related to anxiety (e.g., Eysenck, 1992; Mathews, 1990; Wells & Matthews, 1994; Williams, Mathews, & MacLeod, 1996). Recently, researchers have attempted to modify experimental tests of attention in order to induce selective processing of threat-relevant versus neutral information. In their landmark study, MacLeod and colleagues demonstrated that it was possible to alter attentional biases using a modified probe detection task by adjusting the contingency between threat-relevant (or neutral) cues and a visual probe (MacLeod, Rutherford, Campbell, Ebsworthy & Holker, 2002). Importantly, training attention toward neutral rather than threat cues resulted in diminished emotional responsiveness to a subsequent stressor.

Contemporary cognitive theories propose that socially anxious individuals are hypervigilant to threatening stimuli that relate to concerns about negative evaluation (e.g., Clark & Wells, 1995; Clark, 2001; Rapee & Heimberg, 1997). These theories suggest that selective attention to threat contributes to the persistence of social anxiety by facilitating preferential processing of negative social information, thereby skewing judgments of social events and ultimately preserving fear-relevant beliefs. Empirical evidence regarding attention bias in social anxiety largely comes from research using the probe detection task (MacLeod, Mathews, & Tata, 1986; see Bögels & Mansell, 2004 for a recent review). In the original probe detection task, participants are shown a pair of words for a short time, one above the other, on a computer screen. One of the words is neutral, and the other is threatening. On critical trials (25% of trials), either the upper or lower word is replaced with a dot probe (⋅) and participants are asked to press a button to signal the presence of the probe. Faster detection of the probe following threat-relevant stimuli relative to neutral stimuli is thought to reflect biased attention toward threat.

Because a core feature of GSP is a fear of negative evaluation, studies have used the probe detection task with faces to examine attention bias to threat in social anxiety. In support of cognitive models, several studies have found that participants with high levels of social anxiety and patients diagnosed with social phobia were faster to respond to negative (e.g., angry or disgust) faces relative to neutral faces, implying an attention bias toward threat (Mogg & Bradley, 2002; Mogg, Philippot, & Bradley, 2004; Pishyar, Harris, & Menzies, 2004). For instance, Mogg et al. (2004) found that individuals with social phobia selectively attended to angry faces (versus neutral faces) relative to non-anxious controls. Similarly, Gilboa-Schechtman, Foa, and Amir (1999) found that people with social phobia displayed an attention bias towards negative faces using the face-in-the-crowd paradigm. In contrast, several studies have failed to find evidence of attention biases in social anxiety (e.g., Bradley et al., 1997; Chen, Ehlers, Clark, & Mansell, 2002; Mansell, Clark, Ehlers, & Chen, 1999; Pineles & Mineka, 2005). A review of the extant literature suggests that important methodological differences may have in part accounted for these mixed results. Specifically, studies that have demonstrated an attention bias for negative faces in social anxiety have two features in common: 1) they have used face pairs instead of pairing a face with an object (e.g., chair) in the probe detection task and 2) they have presented the faces for 500 ms or less (see Bogels & Mansell, 2004 for a review). These parameters were used in the current study.

In summary, there is evidence for a relationship between attention bias to threat using the probe detection task and social anxiety. However, because of the correlational nature of these studies, it is not possible to examine the causal nature of this relationship. Conclusions regarding the causal role of attention bias in anxiety can only be drawn from experimental designs in which participants are randomly assigned to conditions, their attention is manipulated, and the effect of this manipulation on anxiety is measured. We now turn to this source of evidence.

MacLeod et al. (2002) conducted such a study by screening a large pool of undergraduate students and selecting those who scored in the middle third of the distribution on a self-report measure of trait anxiety. Participants were then randomly assigned to one of two computerized attention training tasks. One program was designed to train the participants' attention toward threat-relevant words (referred to as the “Attend Threat” condition). The second program was designed to train the participants' attention toward neutral words (referred to as the “Attend Neutral” condition). Both programs resembled the original probe detection task described above. Each program consisted of 672 trials in which pairs of words (one neutral, one threat-relevant) were presented, one above the other, on a computer screen. Word pairs were presented for either 20 ms (subliminal) or 480 ms (supraliminal) intervals. In the Attend Threat condition, probes appeared in the position of the threat word on 576 training trials (93%). The remaining 96 trials were designed to provide a measure of attention bias to threat words. In these test trials, threat word position and probe position were fully crossed as in a typical probe detection task, thus permitting measurement of a participant's tendency to attend preferentially to threat-relevant or neutral words. In the Attend Neutral condition, probes appeared in the position of the neutral word on 93% of the trials, with the remaining 96 trials again providing a measure of attention bias. Following completion of one of the two attention training tasks (i.e., Attend Threat or Attend Neutral) participants were told to rest for four minutes. Finally, the authors manipulated the participants' level of stress by presenting them a series of unsolvable anagrams and telling them that this was an intelligence test. Results revealed that participants in the Attend Threat condition showed faster response latencies for detecting probes following threat words than neutral words. Participants in the Attend Neutral condition showed the opposite pattern of results. However, this pattern was only evident for stimuli that were presented long enough to enter conscious awareness (480 ms presentation). Finally, participants in the Attend Threat condition reported more negative affect in response to the experimental stressor than did those in the Attend Neutral condition. These findings suggested that the attention training procedure influenced participants' emotional vulnerability to a subsequent stressor.

Similar results were obtained by Dandeneau, Baldwin and colleagues using a different attention training procedure (Dandeneau, S., & Baldwin, M. W. (2004). The inhibition of socially rejecting information among people with high versus low self-esteem: The role of attentional bias and the effects of bias reduction training. Journal of Social and Clinical Psychology, 23, 584-602.).

Participants were required to locate a single smiling (accepting) face in a grid of frowning (rejecting) faces. In their initial study, they found that participants with low self esteem who were repeatedly required to find a smiling face among frowning faces later showed a reduced attention bias toward rejection words on an emotional Stroop task relative to participants who completed a control task (Dandeneau & Baldwin, 2004). Subsequent work replicated these findings, and further demonstrated that participants completing the attention training task over several days displayed diminished subjective emotional and physiological responsiveness to a real-life stressor (Dandeneau, Baldwin, Baccus, Sakellaropoulo, & Pruessner, 2007). Specifically, students completing the experimental attention training procedure prior to a final exam reported feeling less stressed and more confident about their exam Similarly, attention training in a group of telemarketers (who routinely experience rejection as part of their work) led to increased self-esteem, lower cortisol levels, lower self-reported stress, higher confidence, and improved work performance. Considered together with the findings of MacLeod et al. (2002), these studies provide the strongest support to date for the hypothesis that individual differences in the allocation of attention to threat-relevant information causally influences one's negative affectivity. At a practical level, these studies suggest that it may be possible to utilize such attention training procedures clinically, that is, training anxious people to direct their attention away from threat information in order to reduce anxiety. Researchers, however, have yet to examine the effects of attention training in a sample of individuals with clinically significant levels of anxiety.

### SUMMARY OF THE INVENTION

The present invention relates to systems and methods for treating patients with a social anxiety disorder. The systems comprise a screen for displaying sets of stimuli, a computer to control the display of stimuli onto the screen during at least one treatment session and a means for the patient to interact with the screen in response to the displayed stimuli. The interaction of the patient with the system during the treatment session is capable of treating patient anxiety associated with social settings.

The present invention also relates to methods of treating anxiety in a patient, with the methods comprising providing an interactive computer program to a subject in need of treatment of anxiety. The interactive programs used in the methods of the present invention are capable of displaying sets of stimuli on a screen to the patient and querying the patient to interact with the screen after the stimuli from each set have been displayed and subsequently removed. The subject is allowed to interact with the interactive program for at least one treatment session, wherein the patient's interaction with the computer program is capable treating the anxiety disorder.

The invention also relates to computer programs capable of being used in the systems and methods of the present invention for treating anxiety. In particular, the invention relates to computer storage media comprising executable code, wherein the executable code is capable of displaying sets of stimuli on a graphical user interface to a user and querying the user to interact with the interface after the stimuli from each set have been displayed and subsequently removed.

### BRIEF DESCRIPTION OF THE DRAWINGS

FIG. 1 is a schematic drawing illustrating an embodiment of the invention. FIG. 1 shows a patient device connected to a remote treatment/assessment computer having modules for delivering image sets to the patient device, collecting timing and location data, calculating attention bias, generating reports, connecting to secure storage, and managing encrypted communications.

FIG. 2 is a schematic drawing illustrating an embodiment of the invention. FIG. 2 shows the operative connections between the device, HIPAA compliant server, and the various modules with their respective databases or data stores.

FIG. 3 is a schematic drawing illustrating an embodiment of the invention. FIG. 3 shows examples of screen images having a fixation symbol supplied by a fixation symbol database, one or more sets of images displayed to the patient device for a specified time and at specified locations on the screen, a patient input indicator connected to a latency monitoring timer and a input location accuracy detector, along with storage databases for collecting the treatment session data, tracking and processing session results, and providing connection and access to a provider system and storage databases.

FIG. 4 is a schematic drawing illustrating an embodiment of the invention. FIG. 4 shows a data flow diagram showing the interaction between a treatment provider, a treatment server, and a patient device.

FIG. 5 is an illustration of a patient and/or provider smart device having the user-end access point developed as an “App” or application.

FIG. 6 is a series of illustrations of screen shots from a patient device showing a welcome screen with login, initial navigation menus, treatment session menus, tracking and history menus, and provider contact information.

FIG. 7 is an illustration of the basic hardware components used in the patient device, the server computer, and the provider device.

FIG. 8 depicts one embodiment of a means for displaying a set of stimuli including a computer, a screen, and an keyboard;

FIG. 9 depicts a treatment session according to one embodiment of the invention, depicting a series of stimuli displayed on a screen for a patient to interact with; and

FIG. 10 depicts a chart showing the results over time of treatment sessions according to one aspect of the invention on individuals in an Attention Modification Program (AMP) or an Attention Control Condition (ACC).

### DETAILED DESCRIPTION OF THE INVENTION

The present invention relates to systems and methods for treating patients with anxiety. In one embodiment, the systems comprise a screen for displaying sets of stimuli, a means for controlling the display of the sets of stimuli, and a means for the patient to interact with the screen in response to the displayed stimuli or commands to the patient. The interaction of the patient with the stimuli relieves anxiety.

In a preferred embodiment, the invention comprises a system comprising a patient device connected to a treatment computer having a pre-assessment module configured to send a pre-assessment instrument to present diagnostic survey questions to the patient device, receive patient inputs from the patient device, and generate a pre-assessment patient profile, said pre-assessment patient profile stored in a secure system memory or database operatively connected to the treatment computer, the treatment computer having a patient profile module configured to analyze the pre-assessment patient profile and communicate to a treatment selection module configured to select one or more treatment sessions by accessing stored treatment sessions from a connected database, the treatment computer having a communication module configured to transmit the one or more treatment sessions to the patient device, and configured to send a pre-assessment profile completion notification to a treatment provider device, the treatment computer having a treatment operations module configured to control delivery of the treatment session and collection data from the treatment session, the treatment session comprises first sending a set of instructions for completing the treatment session to the patient device, and then displaying a fixation symbol for a specific time period on a display of the patient device, the fixation symbol is selected from a connected database of fixation symbols, and then after displaying the fixation symbol, a treatment session consisting of a set of multiple stimuli image pairs is delivered to the patient device, each of the stimuli image pairs are displayed for no more than 500 ms, each stimuli image pair timer period is recorded by the treatment operations module, whereafter the stimuli image pair are removed from the display of the patient device, wherein the stimuli image pairs are re-located from frame to frame where the on-screen display locations of the stimuli image pairs in the previous frame are different from the on-screen display locations of the stimuli image pairs being displayed by the patient device, the treatment operations module configured to receive an input response from the patient device, the input response related with one image of the previously displayed stimuli image pair, the treatment operations module configured to record the length of time it takes from removal of the stimuli image pair from the display of the patient device to the receipt of the input response using an image bias timer, the treatment operations module configured to record the on-screen display location of the input response on the display of the patient device and to analyze on-screen location accuracy of the input response, the treatment computer having a communication module configured to transmit the completed treatment data to a bias calculation module, the bias calculation module is configured to to perform treatment session data analysis using the treatment session data selected from display location of stimuli image pairs, input responses, input response latency, treatment session duration, the treatment computer having a reporting module, and the treatment computer operatively connected to secure file storage.

In a preferred embodiment, the invention comprises a method for calculating an anxiety bias score by measuring latencies between display of stimuli images and patient input, comprising the steps of: (a) generating, on a patient computer having a computer display screen and input means, an electronic pre-assessment patient profile by receiving a series of patient anxiety profile data inputs into an electronic form displayed on a graphical user interface displayed on the computer display screen, said electronic form having anxiety profile questions relating to feelings selected from fear, apprehension, and worry, and questions relating to physiological symptoms selected from heart palpitations, nausea, chest pain, shortness of breath, and headache; (b) saving said electronic pre-assessment patient profile to storage memory on a computer at a remote location to the patient computer, said patient computer having computer program code stored in memory and executable on a processor; (c) providing, on the patient computer, a remotely stored treatment session from a plurality of remotely stored treatment sessions, wherein the remotely stored treatment session is selected by the computer at the remote location operatively connected to the patient computer, said computer at the remote location having computer program code stored in memory and executable on a processor for analyzing the pre-assessment patient profile from the patient computer, and providing the treatment session from the plurality of stored treatment sessions to the patient computer, wherein the treatment session comprises the steps in order of (i) displaying patient instructions on the computer display screen, (ii) displaying a fixation symbol on a central part of the computer display screen for 500 ms or less, (iii) displaying a set of stimuli images on the computer display screen for a display period of 500 ms or less, said stimuli images selected from negative-neutral stimuli images and neutral-neutral stimuli images, said negative-neutral stimuli images comprising a negative stimuli image displayed at a first location on the computer display screen adjacent to a neutral stimuli image displayed at a second location on the computer display screen, and said neutral-neutral stimuli images comprising a neutral stimuli image displayed at the first location on the computer display screen adjacent to a neutral stimuli image displayed at the second location on the computer display screen, (iv) displaying a second symbol at the first or second location on the computer display screen for 500 ms or less, (v) displaying instructions on the computer display screen directing the patient to provide input on the whether the second symbol is displayed at the first or second location, (vi) receiving patient input on the second symbol location, (vii) measuring response latencies between displaying the second symbol and receiving patient input, and saving to storage memory response latencies, patient input, location of the second symbol, and type of stimuli images—negative-neutral stimuli images or neutral-neutral stimuli images, (viii) repeating steps (ii)-(vii) for a length of time selected from a pre-determined length of time, a length of about 20 minutes, and a length of time determined from an analysis of patient responses, wherein the stimuli images from one iteration to the next are different; (d) displaying the treatment session on the computer display screen of the patient computer, wherein the display period is varied based on the response latencies; (e) transmitting to the computer at the remote location the response latencies, the patient input, the location of the second symbol, the type of stimuli images—negative-neutral stimuli images or neutral-neutral stimuli images, and the length of time, and said computer at the remote location having computer program code stored in memory and executable on a processor for calculating an anxiety bias score by subtracting response latencies for negative-neutral stimuli images where the location of the second symbol followed the neutral stimuli image (L-neg), from response latencies for neutral-neutral stimuli images (L-neu), and saving the anxiety bias score to storage memory of said computer at the remote location.

In another preferred embodiment, the invention comprises a process, comprising: providing a system having a means for displaying one or more sets of stimuli to a patient including a computer having one or more processors, a memory, a screen, and an input means, and computer storage media comprising computer programming having executable code, wherein the executable code when executed on the one or processors displays the one or more sets of stimuli on a graphical user interface on the screen to the patient and the computer programming including an interactive computer program that queries the patient to interact with the screen after the one or more sets of stimuli is displayed and subsequently removed, and that is configured to receive a response from the patient on the input means, the one or more sets of stimuli comprising images or words, displaying a first set of stimuli from the one or more sets of stimuli to the patient on the graphical user interface; receiving a set of first responses on the input means from the patient to the first set of stimuli; recording the set of first responses from the patient on the input means to the computer memory and storing the set of first responses for analysis; displaying a second set of stimuli from the one or more sets of stimuli to the patient, the second set of stimuli prompted by the first set of responses to the first set of stimuli, receiving a second set of responses on the input means from the patient to the second set of stimuli; recording the second set of responses from the patient on the input means to the computer memory and storing the second set of responses for analysis; wherein the one or more sets of stimuli include negative stimuli images or words and neutral stimuli images or words, wherein each response of the first set of response and the second set of responses is limited to one of two choices given to the patient; wherein displaying the one or more sets of stimuli comprises first presenting on the display screen a fixation symbol along with an instruction to the patient to focus on the fixation symbol, which subsequently disappears and is replaced by a negative stimuli image or word and a neutral stimuli image or word from the first set of stimuli, the negative stimuli image or word and a neutral stimuli image or word simultaneously displayed around the fixation symbol, along with an instruction to the patient to focus on one of the negative stimuli image or word and a neutral stimuli image or word, wherein the negative stimuli image or word and a neutral stimuli image or word then simultaneously disappear after a display period of 500 milliseconds or less, wherein a probe symbol is displayed where one of the negative stimuli image or word and a neutral stimuli image or word was previously positioned on the graphical user interface, wherein the probe symbol includes an instruction to the patient to enter a response on the input means in a specific manner, the probe symbol remaining on the graphical user interface until the patient enters a response, wherein recording includes the patient response and a response latency, said response latency comprising the amount of time between the display of the probe symbol and the entry of the response by the patient, each said response is one of the first set of responses or the second set of responses, wherein the display period is varied based on the response latency; wherein the probe symbol comprises faces selected from a standardized set of emotional expressions, wherein the standardized set includes faces of eight individuals comprising four male faces and four female faces, the faces of the eight individuals including negative expressions and neutral expressions; analyzing the first set of responses and the second set of responses that are stored in the memory, by comparing the response latency of each response of the first set of response and the response latency of each response of the second set of responses; and calculating a bias score by subtracting response latencies where the probe was a negative expression from response latencies where the probe was a neutral expression; wherein a positive bias score generates a report that the patient has difficulty disengaging attention from a negative stimuli image or word, wherein a negative bias score generates a report that the patient has reduced anxiety.

Any of the embodiments herein may include wherein the computer that controls the display of stimuli is local to the patient or is located at a remote location.

Any of the embodiments herein may include creating a patient profile of the patient by displaying on the graphical user interface a series of questions to assess a level of anxiety in the patient, and obtaining profile answers from the patient using the input means, and storing the patient profile in memory.

Any of the embodiments herein may include wherein the patient profile automatically selects the instructions and the one or more sets of stimuli.

Any of the embodiments herein may include wherein the negative stimuli image or word and a neutral stimuli image or word is displayed in the first set of stimuli or the second set of stimuli or both for a time selected from: at least about 50 ms, at least about 50 ms to about 100 ms, at least about 100 ms to about 200 ms, at least about 200 ms to about 300 ms, at least about 300 ms to about 400 ms, at least about 400 ms to about 500 ms, at least about 500 ms to about 600 ms, at least about 600 ms to about 700 ms, at least about 700 ms to about 800 ms, at least about 800 ms to about 900 ms, at least about 900 ms to about 1 second.

Any of the embodiments herein may include wherein the patient computer comprises one or more user input devices selected from a mouse, a keyboard, a stylus, touch screen, eye or head movement input, or voice command

Any of the embodiments herein may include wherein the location of the stimuli images on the computer display screen is varied based on the response latencies.

Any of the embodiments herein may include wherein the patient profile and the latency data are stored to memory on said computer at the remote location using secure storage technique appropriate to medical information.

Any of the embodiments herein may include wherein the stimuli images are images of people or words and the anxiety disorder is social anxiety disorder.

### Figures

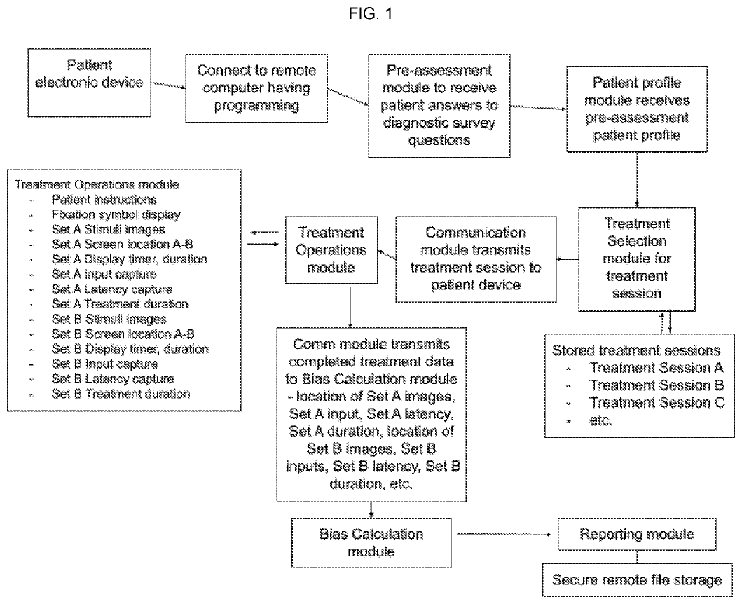

FIG. 1 is a schematic drawing illustrating an embodiment of the invention. FIG. 1 shows a patient device connected to a remote treatment/assessment computer having modules for delivering image sets to the patient device, collecting timing and location data, calculating attention bias, generating reports, connecting to secure storage, and managing encrypted communications.

In one embodiment of the invention, the system connects the patient device to the remote treatment session delivery computer. A pre-assessment module uses a pre-assessment instrument to present diagnostic survey questions to the patient/user. The user/patient inputs their answers and the pre-assessment module generates a pre-assessment patient profile, e.g. high/medium/low anxiety. The series of questions may be used to assess the level of anxiety in a patient before treatment starts. The answers to the questions are stored in a secure system memory or database operatively connected to the remote treatment session delivery computer.

Once the patient has created a patient profile, a treatment session is initiated. In a preferred embodiment, a patient profile module uses an algorithm to analyze the patient profile, e.g. high/medium/low anxiety, to determine which treatment session to display to the patient, or the patient may select a specific session on his/her own.

A treatment selection module accesses stored treatment sessions from a connected databases, and a communication module transmits the treatment session(s) to the patient device. The treatment provider is provided with a notification of the pre-assessment profile and can also select specific treatment sets for use in the treatment session. The system can provide a plurality of treatment sessions which can, in one non-limiting example, be completed in a specific order. In one treatment session, the system first provides the patient with a set of instructions for completing the treatment session. The patient will read the instructions and then begin the treatment session thereafter.

A treatment operations module controls delivery of the treatment session and collection of the data from the treatment session. In one aspect, a treatment session comprises displaying a fixation symbol for a specific time period. The image timer periods are recorded by the system. The fixation symbol may be selected from a database of fixation symbols. After displaying the fixation symbol a set of stimuli image pairs is displayed to the patient. The stimuli are different, for example, one stimuli image may be negative, while the other stimuli image is neutral. Or one stimuli image is positive, while the other is neutral or negative. The patient views both the stimuli for a very brief period of time, where after the stimuli are removed from the display. The pairs of stimuli images are re-located from frame to frame, such that the locations of the images in the previous frame are different from the locations of stimuli images being viewed by the patient.

The patient is then prompted to interact with the system by inputting a response that is correlated with one of the previously displayed stimuli in the set of images. The system records the length of time it takes the patient to input their response using an image bias timer. To modify attention bias or desensitize anxiety, the patient may be instructed to place the input on a blank screen location where the previous screen showed a positive or a neutral image. The system may also record the on-screen location of the input from the patient. The on-screen location accuracy of the input may also be analyzed and reported by the system.

Once the patient provides a response, a second set of stimuli is displayed to the user for a similarly brief period of time, after which the patient is again prompted to input a response. In one aspect, the response is limited to one of two choices given to the patient, such as a left or right click of a mouse, or the input of a specific letter on a keyboard. The treatment session continues as described by briefly displaying a neutral or positive or negative stimuli image followed by a non-matching stimuli image, and then prompting the patient for a response for any amount of time until the session is deemed completed. In one embodiment, the length of the session is typically predetermined by the system, but it could also be determined based on the patient's responses.

A communication module transmits the completed treatment data to a bias calculation module. The bias calculation module uses the treatment session data, e.g. location of Set A images, Set A input, Set A latency, Set A duration, location of Set B images, Set B inputs, Set B latency, Set B duration, etc., to perform treatment session data analysis, and is operatively connected to a reporting module and secure file storage.

### Algorithm

An algorithm may be established and embedded in programming code to receive the various inputs, process the inputs, pull requirements from specific databases, present display graphics for specific times and in specific screen locations, measure and process secondary input latencies and accuracies, generate reporting, and establish ongoing modifications based upon improvements or changes to inputs over time.

Pa—pre-assessment answers and weighting

Pm—profile module

Ts—treatment selection

St—stored treatment modules available

To—treatment operation

Fx—fixation symbol data

Tx—transmitted data

Loc—location of images data

Pi—patient input data

Pl—patient input latency measurement data

Td—treatment duration data

Bx—bias calculation data

Rv—provider treatment revision data

Tsn—follow up treatment selection

Tn—follow up treatment operation

To=Fx+Tx+Bx

Tx=Loc+Pi+Pl+Td

Rv=To+Bx

Tsn=To+Rv

Tn=Fx+Tsn

The data relationships listed above are non-limiting examples. Combinations of variables are used in accordance with the teaching herein to obtain and generate treatment data and treatment regimens.

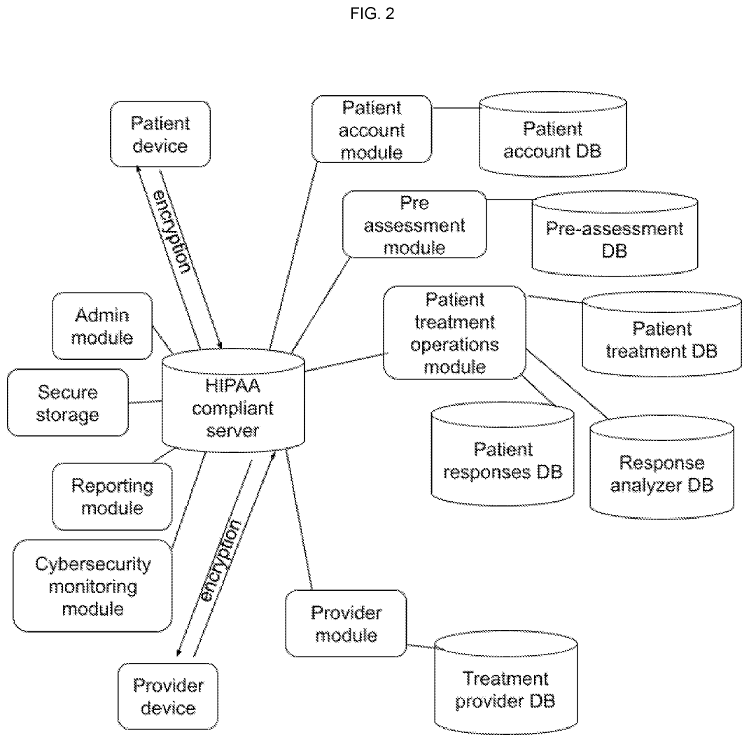

FIG. 2 is a schematic drawing illustrating an embodiment of the invention. FIG. 2 shows the operative connections between the device, HIPAA compliant server, and the various modules with their respective databases or data stores. FIG. 2 shows the HIPAA compliant server having an admin module, a reporting module, and a cybersecurity monitoring module. The HIPAA server is also operatively connected to secure storage. Encrypted communications are established between the patient device and the provider device. The server also includes a patient account module connected to a patient account data storage, and a pre-assessment module connected to a pre-assessment data storage facility. A patient treatment operations module is connected to a patient treatment data storage facility, a patient responses storage, and a response analyzer database. The server also has a provider module that is operatively connected to the treatment provider data storage, and provides access by the treatment provider for the control needed to deliver treatments sessions and obtain and analyze reports from the patient treatment sessions.

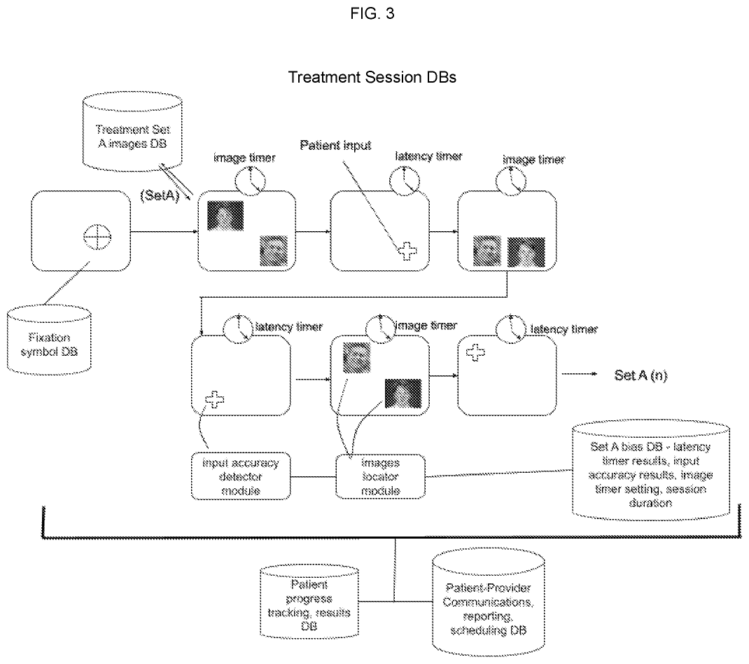

FIG. 3 is a schematic drawing illustrating an embodiment of the invention. FIG. 3 shows examples of screen images having a fixation symbol supplied by a fixation symbol database, one or more sets of images displayed to the patient device for a specified time and at specified locations on the screen, a patient input indicator connected to a latency monitoring timer and a input location accuracy detector, along with storage databases for collecting the treatment session data, tracking and processing session results, and providing connection and access to a provider system and storage databases.

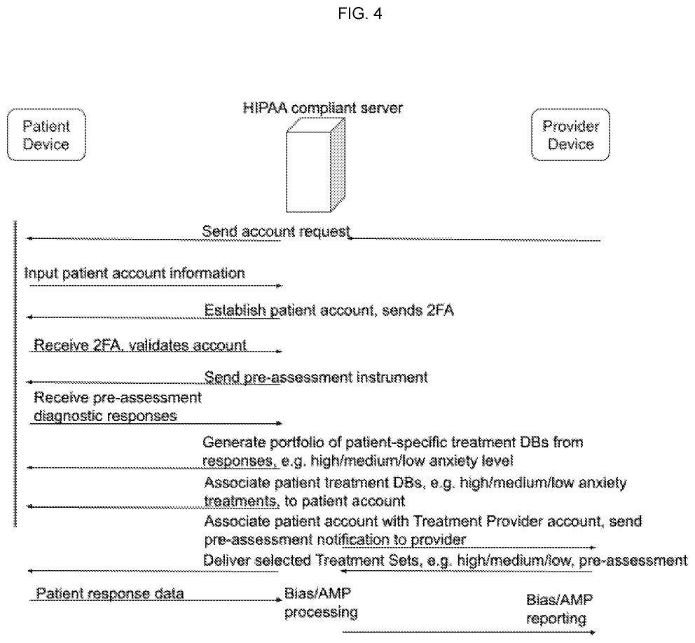

FIG. 4 is a schematic drawing illustrating an embodiment of the invention. FIG. 4 shows a data flow diagram showing the interaction between a treatment provider, a treatment server, and a patient device. FIG. 4 starts with a request from the provider through the server to the patient device. The patient is prompted to input patient account information and establish a patient account. The server confirms the patient account and sets up security such as two-factor authentication to validate the account. The pre-assessment instrument is delivered from the server to the patient device. The patient completes the pre-assessment instrument and the server receives the pre-assessment diagnostic responses. The server then generates a portfolio of patient-specific treatment sessions from the results of the pre-assessment. As an non-limiting example, the patient pre-assessment may show a patient to have high/medium/low anxiety and treatment sessions are chosen from a pool of treatment sessions that match the pre-assessment anxiety level of the patient, e.g. high/medium/low. The server associates the patient account with the provider account and sends a notification to the provider device that the pre-assessment was completed and including the pre-assessment results. Treatment image sets are then delivered to the patient. The image sets may be selected by the treatment provider, may be selected automatically by the server, or may also be selected by the patient. The patient response data is collected, e.g. location of Set A images, Set A input, Set A latency, Set A duration, location of Set B images, Set B inputs, Set B latency, Set B duration, etc., and sent to the server for processing. The server may also send reports to the provider device.

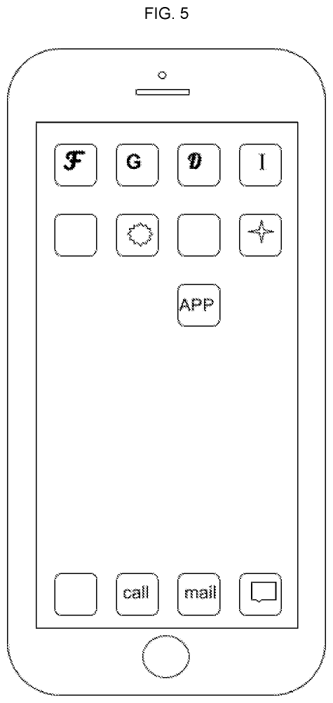

FIG. 5 is an illustration of a patient and/or provider smart device having the user-end access point developed as an “App” or application.

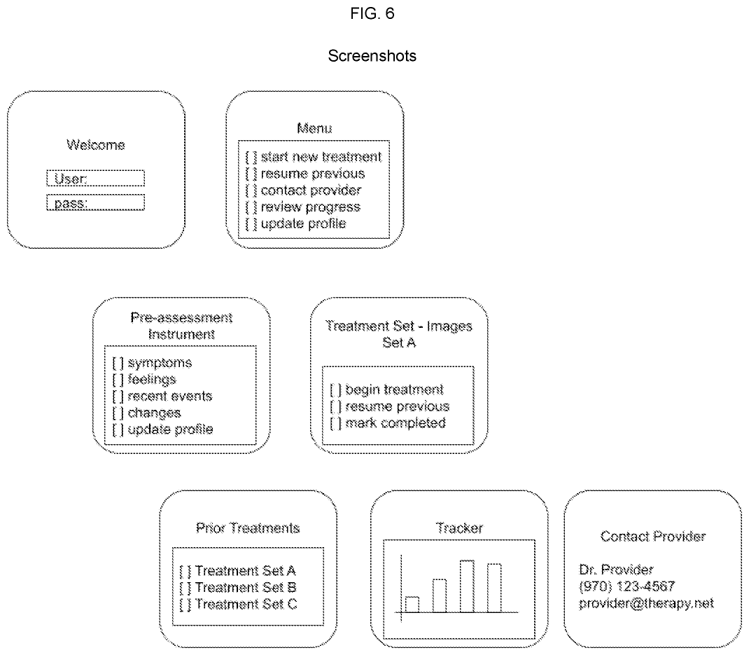

FIG. 6 is a series of illustrations of screen shots from a patient device showing a welcome screen with login, initial navigation menus, treatment session menus, tracking and history menus, and provider contact information. FIG. 6 shows that a welcome screen may include a username and password login. This may also be followed up with a 2FA challenge. A menu is presented to the patient to start a new treatment and may include options for resume previous treatment, contact the provider, review patient progress, and/or update the pre-assessment profile. An example of a secondary menu may include a pre-assessment instrument menu for collecting pre-assessment data and asking the patient about symptoms, feelings, recent events, changes, and updates to their profile. An example of another menu may include a Treatment Set—Images Set A menu to begin treatment, resume previous, or mark completed. Additional menu examples may include a Prior Treatments menu, a Progress Tracker menu, and/or a Contact Provider menu.

### Hardware Overview

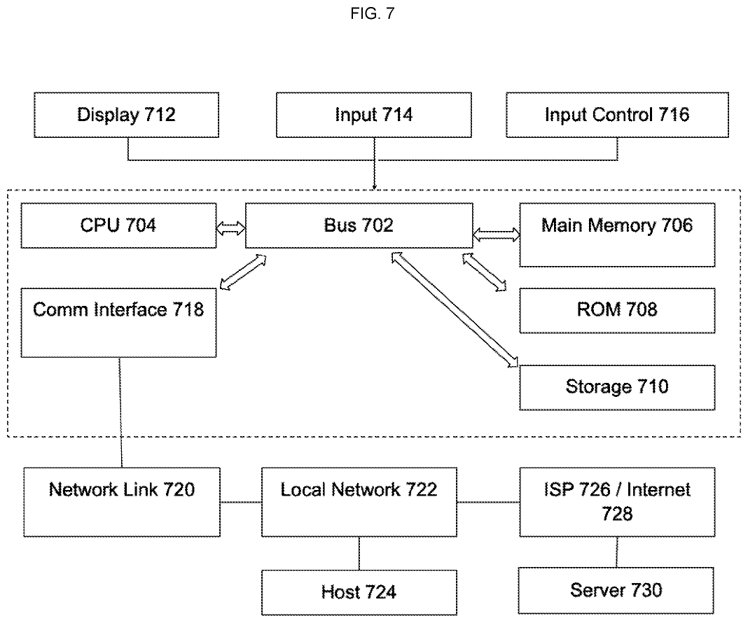

FIG. 7 is an illustration of the basic hardware components used in the patient device, the server computer, and the provider device. According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

For example, FIG. 7 is a block diagram that illustrates a computer system 700 upon which an embodiment of the invention may be implemented. Computer system 700 includes a bus 702 or other communication mechanism for communicating information, and a hardware processor 704 coupled with bus 702 for processing information. Hardware processor 704 may be, for example, a general purpose microprocessor.

Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Such instructions, when stored in non-transitory storage media accessible to processor 704, render computer system 700 into a special-purpose machine that is customized to perform the operations specified in the instructions.

Computer system 700 further includes a read only memory (ROM) 708 or other static storage device coupled to bus 702 for storing static information and instructions for processor 704. A storage device 710, such as a magnetic disk or optical disk, is provided and coupled to bus 702 for storing information and instructions.

Computer system 700 may be coupled via bus 702 to a display 712, such as an OLED, LED-LCD, cathode ray tube (CRT), etc., for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane.

Computer system 700 may implement the techniques described herein using customized hard-wired logic, one or more ASICs or FPGAs, firmware and/or program logic which in combination with the computer system causes or programs computer system 700 to be a special-purpose machine. According to one embodiment, the techniques herein are performed by computer system 700 in response to processor 704 executing one or more sequences of one or more instructions contained in main memory 706. Such instructions may be read into main memory 706 from another storage medium, such as storage device 710. Execution of the sequences of instructions contained in main memory 706 causes processor 704 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage device 710. Volatile media includes dynamic memory, such as main memory 706. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 702. Transmission media can also take the form of acoustic or light waves, such as those generated during radio-wave and infra-red data communications.

Various forms of media may be involved in carrying one or more sequences of one or more instructions to processor 704 for execution. For example, the instructions may initially be carried on a magnetic disk or solid state drive of a remote computer. The remote computer can load the instructions into its dynamic memory and send the instructions over a telephone line using a modem. A modem local to computer system 700 can receive the data on the telephone line and use an infra-red transmitter to convert the data to an infra-red signal. An infra-red detector can receive the data carried in the infra-red signal and appropriate circuitry can place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card, cable modem, satellite modem, or a modem to provide a data communication connection to a corresponding type of telephone line. As another example, communication interface 718 may be a local area network (LAN) card to provide a data communication connection to a compatible LAN. Wireless links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the “Internet” 728. Local network 722 and Internet 728 both use electrical, electromagnetic or optical signals that carry digital data streams. The signals through the various networks and the signals on network link 720 and through communication interface 718, which carry the digital data to and from computer system 700, are example forms of transmission media.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718.

The received code may be executed by processor 704 as it is received, and/or stored in storage device 710, or other non-volatile storage for later execution.

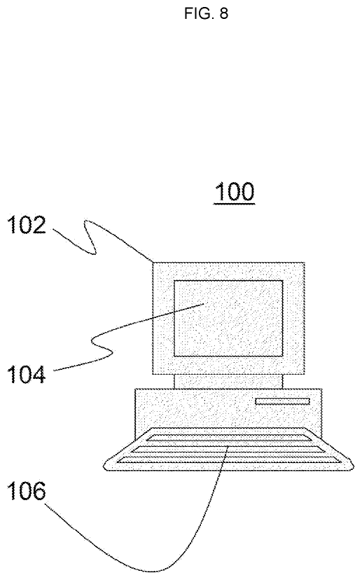

FIG. 8 represents one embodiment of the system 100 of the invention. The system of FIG. 8 comprises a means for displaying sets of stimuli, such as a computer 102, a screen 104 on which the sets of stimuli are displayed, and a tool for interacting with the screen, such as a keyboard 106. Additional examples of means for displaying sets of stimuli to a patient or user include, but are not limited to, human intervention, for example a therapist physically displaying the stimuli to the patient. As used herein, a computer is defined as it would be used in the computer sciences arts. The computer may comprise one or more processors, a memory, a storage device, a display, and an input means.

In one aspect, the system uses sets of stimuli, such as images or words, to prompt a response by the patient. The patient's response to the stimuli is recorded by the system and stored for analysis and further treatment. The system may then prompt the patient with another set of stimuli based on their response. The stages of presenting sets of stimuli to the patient, recording the patient response, and providing another set of stimuli can be repeated as needed. The system is capable of analyzing the patient response and presenting the patient with new sets of stimuli that aid in the relief of anxiety.

In one embodiment of the invention, the system first probes a patient to create a patient profile of the patient. The probes include a series of questions to assess the level of anxiety in a patient before treatment starts. The answers to the questions are stored in a system memory.

Once the patient has created a patient profile, a treatment session is initiated. An algorithm may analyze the patient profile to determine which treatment session to display to the patient, or the patient may select a specific session on his/her own. The system can provide a plurality of treatment sessions which can, in one non-limiting example, be completed in a specific order. In one treatment session, the system first provides the patient with a set of instructions for completing the treatment session. The patient will read the instructions and then begin the treatment session thereafter.

In one aspect, a treatment session comprises displaying a set of stimuli to the patient. One stimuli is negative, while the other stimuli is neutral. The patient views both the stimuli for a brief period of time, where after the stimuli are removed from the display. The patient is then prompted to interact with the system by inputting a response that is correlated with one of the previously displayed stimuli in the set of images. Once the patient provides a response, a second set of stimuli is displayed to the user for a similarly brief period of time, after which the patient is again prompted to input a response. In one aspect, the response is limited to one of two choices given to the patient, such as a left or right click of a mouse, or the input of a specific letter on a keyboard. The treatment session continues as described by briefly displaying a neutral stimuli and negative stimuli, and then prompting the patient for a response for any amount of time until the session is deemed completed. In one embodiment, the length of the session is typically predetermined by the system, but it could also be determined based on the patient's responses.

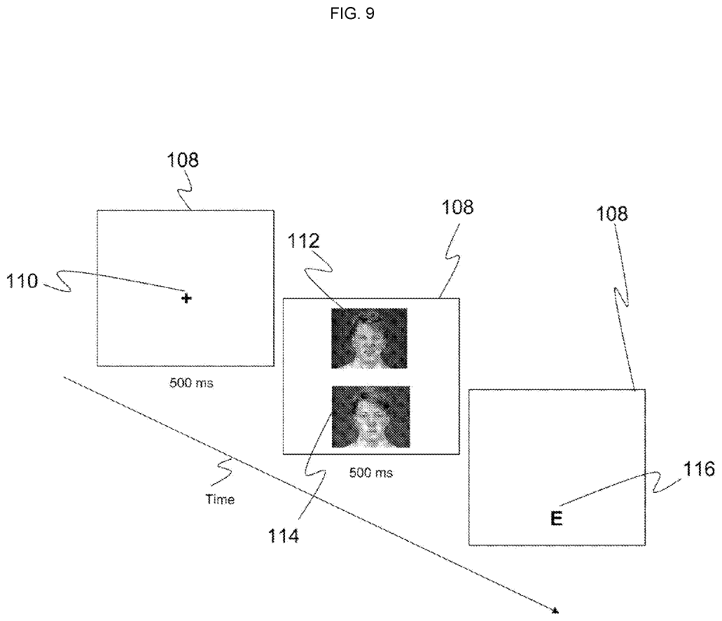

In FIG. 9, one embodiment representing a system for treating patients with social anxiety, the set of stimuli displayed comprise images of people, such as photographs, is depicted. The patient is first presented with a display screen 108 without the images, containing only a first symbol 110 positioned between the images to be later displayed. In the embodiment shown in FIG. 9, the first symbol 110 is a fixation cross. The patient is asked to focus on the fixation symbol, which subsequently disappears and is replaced by two images simultaneously displayed above and below the symbol 110 sign as a top image 112 and a bottom image 114. The patient is instructed to focus on one of the images, such as the top image 112, for the first set of images displayed. In the embodiment shown in FIG. 9, the top image 112 is a negative stimuli, and shows a person with a negative facial expression, while the bottom image 114 is a neutral stimuli, and shows a person with a neutral or emotionless expression. The images then simultaneously disappear after a short period of time—approximately 500 milliseconds in one embodiment—and a second symbol, or probe 116 appears where one of the images was previously positioned. The probe 116 corresponds to a response that the patient is instructed to provide. In the embodiment of FIG. 9, the probe is either the letter “E” or the letter “F.” The patient is instructed to respond in a specific manner depending on which probe 116 appears—if the “E” appears, the patient can press the left button on a mouse, and if an “F” appears, the patient can press the right button on the mouse.

The system, through the selection of specific stimuli and the placement of the probe 116, helps to train the patient to focus away from the negative stimuli, thereby eliminating attention bias and leading to the reduction in anxiety associated with an anxiety disorder through methods that will be described in more detail below.

In one aspect of the invention, the patient is provided with instructions prior to beginning the therapy session; however, in another aspect, the instructions can be provided during the treatment session.

In another embodiment of the system for treating patients with social anxiety, the images of people are replaced with words. As with the images, two words are displayed simultaneously, with one word having a negative connotation and the other word having a neutral connotation. The patient is then prompted to interact with the system as described before—by responding in a specific manner depending on which symbol appears after the words are displayed.

One skilled in the art will appreciate that the set of stimuli is not limited to two, and a plurality of images or words may be presented to the user to effectuate a therapeutic result.

Once the patient completes a therapy session, the patient's interactions with the system during the therapy session are stored in the system memory. The stored interactions can then be accessed by a health professional, such as the patient's therapist, for analysis. The system can also use the stored interactions to determine the next treatment session that should be presented to the patient. This determination is based on an algorithm that utilizes the stored interactions to determine a proper treatment session to present to the patient. Additionally, in alternative embodiments, the patient or the health professional can select the next treatment session. The selection can be done at the computer or display screen itself, or remotely, such as a health professional who receives the stored interaction data at a remote location and then transmits back instructions to the system or the patient for which treatment session to perform next.

The patient can interact with the system through a variety of devices, including but not limited to a mouse, keyboard, stylus, pen light, or laser. Additionally, the system can be configured to respond to the patient's touch, such as with a touch screen display device, or to respond to the patient's eye movement, head movement, or voice. Additional software and hardware may be needed to accomplish the various methods of interacting with the system described herein.

In one embodiment, the user or the algorithm can change the time during which the images are displayed. In specific embodiments, the images are displayed at least about 50 ms, at least about 50 ms to about 100 ms, at least about 100 ms to about 200 ms, at least about 200 ms to about 300 ms, at least about 300 ms to about 400 ms, at least about 400 ms to about 500 ms, at least about 500 ms to about 600 ms, at least about 600 ms to about 700 ms, at least about 700 ms to about 800 ms, at least about 800 ms to about 900 ms, at least about 900 ms to about 1 second. In one specific embodiment, the images are displayed about 500 ms.

The system also comprises numerous self-assessments and evaluation tools for the patient to keep track of his/her progress during the treatments. The tools include, in one aspect, electronic forms to fill out to evaluate the level of perceived anxiety, or questions to answer regarding the level of anxiety felt. The questions and forms are displayed, in one aspect, on the computer display by the computer that is running the system. The information gathered from these additional tools is also stored in the system and can be used by a healthcare professional or a therapist to review the patient's progress. Additionally, the system can assess the data on its own using algorithms to measure and display progress, and show the patient or therapist the progress that is being made.

### I. Study

In one embodiment, a study was conducted using a 2 (Group)×2 (Time: pre-test, post-test) mixed design. Participants were randomly assigned to an Attention Modification Program (AMP, n=17) or an Attention Control Condition (ACC, n=17). They were assessed using self-report and interviewer measures before and after eight sessions of training Additionally, all participants in the AMP condition were assessed once more approximately 4 months after the post-assessment in order to examine the longevity of any symptom change.

The faces used in this probe detection task were selected from a standardized set of emotional expressions (Matsumoto, D. & Ekman, P. (1989). The Japanese and Caucasian Facial Expressions of Emotion (JACFEE) and Neutrals (JACNeuF). San Francisco, Calif.: Intercultural and Emotion Research Laboratory, Department of Psychology, San Francisco State University). The set includes eight individuals (four male, four female) displaying disgust (or negative) expressions, and neutral expressions.

### Attention Modification Program (AMP)

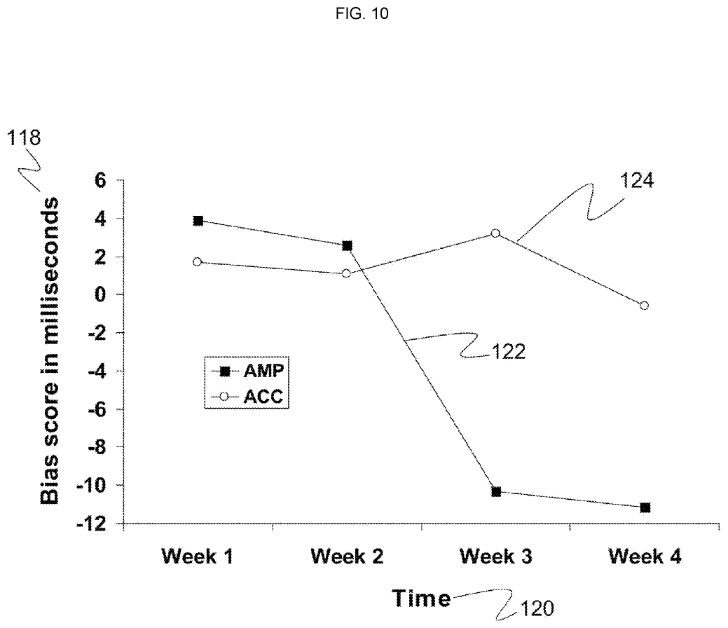

Participants assigned to the AMP condition received a computer delivered attention training protocol. The AMP protocol included eight 20-minute sessions delivered over a four week period (i.e., twice weekly sessions). During each session, as illustrated in FIG. 9, participants completed a probe detection task that began with a fixation cross 110 presented for 500 ms. The computer then presented participants two faces of the same individual, one face on top 112 and one on bottom 114, with combinations of two emotions (i.e., neutral and disgust, or neutral and neutral). After 500 ms, a probe 116 (either the letter “E” or the letter “F”) appeared in the location of one of the two faces. The participants were instructed to decide if the letter was an E or an F by pressing the corresponding button (left or right) on the computer mouse. The probe 116 remained on the screen 108 until participants responded. The next trial began as soon as participants responded. Participants were told that it was important that they perform the task as quickly as possible without sacrificing accuracy. In previous research using this paradigm, it was found that participants' average accuracy to be 95% or higher. The results of one trial are illustrated in the chart in FIG. 10, where the bias score 118 over time 120 of the group of AMP participants 122 is plotted against the bias score of the group of ACC participants 124. The chart clearly shows the reduction in attention bias amongst the AMP group participants 122 over a period of four weeks, with the most significant drop between Week 2 and Week 3.

During each session, AMP participants saw 160 trials that consisted of various combinations of probe type, probe position, emotion type, and face (four male and four female faces). Of the 160 trials, 128 trials (i.e., 80% of trials) included one neutral face and one disgust face [2 (E or F)×2 (top or bottom)×8 (faces)×4 (repetition). During these neutral-disgust trials the probe always replaced the neutral face. The remaining 32 trials (i.e., 20% of trials) included only neutral faces [2 (E or F)×2 (top or bottom)×8 (faces). Thus, although there was no specific instruction to direct attention away from disgust faces, on 80% of all trials the position of the disgust face determined the position of the probe (i.e., in the location opposite the disgust face).

### Attention Control Condition (ACC)

The ACC was identical to the AMP procedure except that during the presentation of the trials where a disgust face was present (i.e., neutral-disgust trials), the probe appeared with equal frequency in the position of the disgust face and the neutral face. Therefore, of the 160 trials, 64 trials (i.e., 40% of trials) were neutral-disgust with the probe following the disgust face, 64 trials (i.e., 40% of trials) were neutral-disgust with the probe following the neutral face, and the remaining 32 trials (i.e., 20% of trials) included only neutral faces as in the AMP. Thus, neither the disgust face nor neutral face had signal value regarding the position of the probe.

After completing eight sessions of AMP or ACC, participants completed a post-assessment identical to the pre-assessment. Finally, participants in the AMP group were invited to complete follow-up assessments, which occurred approximately 4 months later.

### Attention Bias Index

To examine the effect of the computerized training on participants' attention bias, response latencies were compared for trials that consisted of two neutral stimuli (N-N trials) with response latencies for trials that comprised one neutral and one disgust face (N-D trials). Enhanced ability to disengage attention from threat would result in faster response latencies when responding to a probe following a neutral face in the N-D trials compared to responding to a probe following a neutral face in the N-N trials (see Koster, Crombez, Verschuere, & Houwer, 2004). Participants in the AMP group saw twice as many N-D trials with the probe following the neutral face than those in the ACC because of the nature of our training contingency. Therefore, to compare the same number of response latencies across groups the results were compared for the first 16 trials of this type in each group. These values were compared to the N-N trials (16 trials) from both groups.

### Statistical Analyses

Consistent with previous treatment outcome studies of social anxiety, analyses of covariance (ANCOVAs) were conducted on post-treatment scores, controlling for pre-treatment scores (e.g., Heimberg, R. G., Liebowitz, M. R., Hope, D. A., Schneier, F. R., Holt, C. S., Welkowitz, L. A., et al. (1998). Cognitive behavioral group therapy vs. Phenelzine therapy for social phobia. Archives of General Psychiatry, 55, 1133-1141).

### Results

To ensure that random assignment did not create groups differing in demographics or measures of anxiety and depression at pre-treatment, t-tests were conducted comparing groups at pre-treatment on various measures. This analysis revealed that groups did not differ significantly at pre-treatment on any of the measures (ps>0.2).

### Interviewer Measures

At post-treatment, the AMP group had significantly lower scores than the ACC group on the Liebowitz Social Anxiety Scale (LSAS), F(1, 31)=14.15, p<0.001, when pre-treatment scores were partialled out. Moreover, the AMP group had significantly lower functional impairment scores at post-treatment compared to the ACC group on the Sheehan Disability Scale, F(1,31)=5.38, p<0.03, when pre-treatment scores were partialled out. However, groups did not differ on their Hamilton depression scores at post-treatment controlling for pre-treatment scores, F(1,31)=0.25, p=0.46.

Diagnostic status after treatment was also examined. These data revealed that a significantly higher proportion of the participants in the AMP group (44%) no longer met diagnostic criteria for social phobia compared to the ACC group (13%), X2(1)=4.03, p<0.05.

### Self-Report Measures

At post-treatment, the AMP group had significantly lower scores than the ACC group on the Social Phobia and Anxiety Inventory (SPAI), F(1, 31)=4.14, p<0.05, when pre-treatment scores were partialled out. However, groups did not differ on their BDI [F(1, 31)=0.08, p=0.77, STAI-S, [F(1, 31)=0.29, p=0.59, or STAI-T [F(1, 31)=0.37, p=0.55 at post-treatment controlling for pre-treatment scores.

### Magnitude of Effect

To examine the magnitude of improvement in symptoms, effect sizes were calculated (d=pre score−post score/pooled standard deviation) for each group. In line with Cohen's (1988) suggestion, the effect size for the LSAS in the AMP group was classified as large, d=1.32 (Cohen, J. (1988). Statistical power analysis for the behavioral sciences. (2nd ed.). New York: Lawrence Erlbaum Associates). The effect size for the ACC group was small, d=0.43.

### Follow-Up

Follow-up data was collected for four months following the termination of the training sessions for participants assigned to the AMP group. 16 follow-up assessments for 17 participants (94%) in the AMP group were collected. Scores on most interviewer measures as well as self-report measures show a downward trend. Participants' LSAS scores at follow-up were not significantly different from their post, t(15)=1.56, p=0.10. However, participants' LSAS scores at follow-up were significantly lower than their pre-treatment scores, t(15)=5.20, p<0.001 Similarly, participants' SPAI scores at follow-up were not significantly different from their post, t(15)=1.15, p=0.27. However, participants' SPAI scores at follow-up were significantly lower than their pre-treatment scores, t(15)=3.30, p<0.01. Thus, the decrease in symptoms after training appears to be a lasting effect.

### Change in Attention Bias

Participants' response latencies on the probe detection task were submitted to a 2 (Group: AMP, ACC)×2 (Trial type: neutral-neutral, neutral-disgust)×4 (Week: one, two, three, four) ANOVA with repeated measurement on the last two factors. This analysis revealed a significant main effect of Week [F(3, 96)=41.14, p<0.01 that was modified by a significant interaction of Group X Week X Trial type [F(3, 96)=5.60, p<0.02. None of the other effects were significant (ps>0.2). To explore this 3-way interaction further, separate 4 (Week)×2 (Trial Type) ANOVAs were conducted in the AMP and the ACC group.

To simplify the analysis a bias score was calculated by subtracting participants' response latencies for neutral-disgust trials where the probe followed the neutral face from response latencies for neutral-neutral trials. To ensure that the same number of trials was used for both groups, only the first 16 neutral-disgust trials were included in each group (see Procedure section). A positive score on this index implies difficulty disengaging attention from threat.

These bias scores were submitted to a 2 Group×4 Week (1, 2, 3, 4) ANOVA with repeated measurement on the second factor. These analyses revealed a main effect of Week [F(3,96)=8.23, p<0.007 that was modified by an interaction of Group X Week [F(3, 96)=5.99, p<0.02. The main effect of Group was not significant [F(1, 32)=1.80, p=0.19. To probe this interaction, a simple effects analysis was conducted. Simple effect of Group revealed that the AMP and the ACC did not differ in their bias score during week 1 [t(32)=0.32, p=0.75 or week 2 [t(32)=0.22, p=0.82. However, participants in the AMP had significantly lower bias scores than the ACC group during week 3 [t(32)=2.29, p<0.03 and week 4 [t(32)=2.63, p<0.01. Simple effect of Week revealed that in the AMP group there was significant linear decrease in bias scores across time [F(1, 16)=8.67, p<0.01. However, the ACC group did show a change in bias across time [F(1,16)=8.23, p<0.007. These data are depicted in FIG. 10.

### Correlational Analyses

Group membership was correlated with attention bias during week 4 (r=0.42, p<0.01). Moreover, attention bias during week 4 was correlated with change in anxiety on the LSAS (r=0.34, p<0.05).

### Discussion

As predicted, attention training successfully changed attention to threat, as well as symptoms of social anxiety in individuals diagnosed with Generalized Social Phobia. At post-treatment, independent assessors rated participants completing attention training as significantly less socially anxious and less functionally impaired than the control group. Further, participants' self-report of social anxiety symptoms corroborated interviewer ratings. Finally, 44% of participants in the AMP condition no longer met DSM-IV criteria for social phobia after training, compared to 13% of participants in the control condition.

Information processing measures corroborated the findings from interviewer and self-report measures. Specifically, the AMP group showed a decrease in bias for threat over the course of the study. The present work expands the extant literature, however, in serving as the first study to successfully manipulate attention processes using a computerized procedure in a sample of clinically anxious patients.

Several explanations may account for the reduction in social anxiety associated with attention training. Previous research indicates that socially anxious individuals preferentially process negative social information (e.g., Gilboa-Schechtman et al., 1999; Veljaca & Rapee, 1998). To the extent that attention biases toward threat are causally involved in the maintenance of anxiety (e.g., MacLeod et al., 2002), then any procedure that normalizes this bias would be expected to also reduce anxiety symptoms. Consistent with this hypothesis, participants in the AMP group displayed a reduction in attention bias to threat-relevant cues over the course of treatment. In keeping with findings from previous work, it may be that the attention training procedure reduced participants' emotional vulnerability in the context of real-life social encounters (e.g., Dandeneau et al., 2007; MacLeod et al., 2002).

The current research represents the first study to assess the long-term impact of an attention training procedure on anxiety. Follow-up assessments revealed that participants maintained symptom reduction for 4 months on average after completing the training Notably, this maintenance of gains occurred in the absence of booster sessions or further contact. Those findings suggested that the beneficial effects of the attention training program were enduring.

The system described herein is discussed with relevance to the treatment of social anxiety, but treatment of other anxiety-related disorders is also possible. This includes, but is not limited to, post-traumatic stress disorder (PTSD), general anxiety disorder, panic disorder, and social anxiety disorder.

### II. Computer Program Aspects

The computer where the system resides may also comprise a main memory, a random access memory (RAM), and, optionally a secondary memory. In the computer used to implement the method and programs of the present invention, storage for the programs is provided by the main memory and/or the secondary memory.

Examples of secondary memories include, but are not limited to, for example, a hard disk drive and/or a removable storage drive, representing a floppy disk drive, a magnetic tape drive, a compact disk drive, a DVD drive, a flash drive, etc. The removable storage drive may read from and/or write to a removable storage unit in a well-known manner.

Removable storage unit, also called a program storage device or a computer program product, represents a floppy disk, magnetic tape, compact disk, a DVD, a flash drive, etc. As will be appreciated, the removable storage unit may also comprise a computer usable storage medium having stored therein computer software (programs) and/or data.

Computer programs can be stored in main memory and/or the secondary memory. Such computer programs include, for example, computer programs corresponding to the applications. These computer programs, when executed in their respective computers, enable the processors in those computers to perform the methods and features of the present invention. Accordingly, such computer programs represent controllers of their respective computers.

In one aspect of the invention, the computer that controls the display of stimuli is local to the patient, but in another aspect, the computer can be located at a remote location, such that the patient only has access to the display and a means for interacting with the computer, such as a mouse or keyboard. The use of a remote computer may be suited for a study where a patient is in a different location than a therapist, so that the patient may undergo a treatment session while the computer is in a location with the therapist who is more frequently accessing and reviewing the patient's stored interactions and patient profile. The patient profile may also be more secure on a remote computer as opposed to a computer that is local to the patient.

In another embodiment, the screen on which the stimuli are displayed can be a computer monitor. Namely, a computer to control the display of stimuli onto the screen during at least one treatment session and a means for the patient to interact with the screen in response to the displayed stimuli. The interaction of the patient with the system during the treatment session is capable of treating patient anxiety associated with social settings.

The present invention also relates to methods of treating an anxiety disorder in a patient, with the methods comprising providing an interactive computer program to a subject in need of treatment of anxiety. The interactive programs used in the methods of the present invention are capable of displaying sets of stimuli on a screen to the patient and querying the patient to interact with the screen after the stimuli from each set have been displayed and subsequently removed. The subject is allowed to interact with the interactive program for at least one treatment session, wherein the patient's interaction with the computer program is capable of treating the anxiety disorder.

The invention also relates to computer programs capable of being used in the systems and methods of the present invention for treating anxiety. In particular, the invention relates to computer storage media comprising executable code, wherein the executable code is capable of displaying sets of stimuli on a graphical user interface to a user and querying the user to interact with the interface after the stimuli from each set have been displayed and subsequently removed.

The present invention may be implemented using hardware, software or a combination thereof and may be implemented in a computer system or other processing system. Various software implementations are described in terms of this exemplary computer system. After reading this description, it will become apparent to a person skilled in the relevant art how to implement the invention using other computer systems and/or computer architectures.

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited, unless so stated, to particular methods, order of processing steps, components, materials, systems, partial aspects of processes, components or systems, uses, compounds, compositions, standards, routines, modes, computers, hardware, firmware, and software programming which can, of course, vary. It is also to be understood that the terminology used herein is for the purpose of describing particular embodiments only, and is not intended to be limiting unless specifically stated.

While various embodiments have been described above, it should be understood that they have been presented by way of example only, and not limitation. Where methods described above indicate events occurring in specific order, the ordering of events is sequential, but the invention contemplated herein may also include modifications that do not depart from the scope and spirit of the invention. Additionally, certain of the events may be performed concurrently in a parallel process when possible, as well as performed sequentially as described above.

Where schematics and/or embodiments described above indicate certain components arranged in certain orientations or positions, the arrangement of components is specified, but the invention contemplated herein may also include modifications that do not depart from the scope and spirit of the invention.

While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described that do not depart from the scope and spirit of the invention. Various of the above-disclosed and other features and functions, or alternatives thereof, may be combined into many other different systems or applications that do not depart from the scope and spirit of the invention.

Various presently unforeseen or unanticipated alternatives, modifications, variations, or improvements therein may be subsequently made by those skilled in the art, each of which is also intended to be encompassed by the disclosed embodiments. It is therefore to be understood that within the novel, unobvious, enabled, and described scope of the broadest reasonable interpretation of the appended claims, the invention may be practiced otherwise than as narrowly described. Accordingly, all such modifications are intended to be included within the novel, unobvious, enabled, and described scope of this invention as defined in the broadest reasonable interpretation of the following claims.

1. A system for treating patients with an anxiety disorder, the system comprising:
a screen for displaying sets of stimuli,
a computer to control the display of stimuli onto the screen during at least one treatment session,
a means for the patient to interact with the screen in response to the displayed stimuli,
wherein the interaction during the treatment session relieves patient anxiety associated with an anxiety disorder.

2. A computer program product for treating patients with an anxiety disorder, comprising program code means which when executed by a computer carry out the following steps:
displaying sets of stimuli on a graphical user interface to a user; and
querying the user to interact with the interface after the stimuli from each set have been displayed and subsequently removed, wherein the interaction relieves patient anxiety associated with an anxiety disorder.

3. A system, comprising: a patient device connected to a treatment computer having a pre-assessment module configured to send a pre-assessment instrument to present diagnostic survey questions to the patient device, receive patient inputs from the patient device, and generate a pre-assessment patient profile, said pre-assessment patient profile stored in a secure system memory or database operatively connected to the treatment computer, the treatment computer having a patient profile module configured to analyze the pre-assessment patient profile and communicate to a treatment selection module configured to select one or more treatment sessions by accessing stored treatment sessions from a connected database, the treatment computer having a communication module configured to transmit the one or more treatment sessions to the patient device, and configured to send a pre-assessment profile completion notification to a treatment provider device, the treatment computer having a treatment operations module configured to control delivery of the treatment session and collection data from the treatment session, the treatment session comprises first sending a set of instructions for completing the treatment session to the patient device, and then displaying a fixation symbol for a specific time period on a display of the patient device, the fixation symbol is selected from a connected database of fixation symbols, and then after displaying the fixation symbol, a treatment session consisting of a set of multiple stimuli image pairs is delivered to the patient device, each of the stimuli image pairs are displayed for no more than 500 ms, each stimuli image pair timer period is recorded by the treatment operations module, whereafter the stimuli image pair are removed from the display of the patient device, wherein the stimuli image pairs are re-located from frame to frame where the on-screen display locations of the stimuli image pairs in the previous frame are different from the on-screen display locations of the stimuli image pairs being displayed by the patient device, the treatment operations module configured to receive an input response from the patient device, the input response related with one image of the previously displayed stimuli image pair, the treatment operations module configured to record the length of time it takes from removal of the stimuli image pair from the display of the patient device to the receipt of the input response using an image bias timer, the treatment operations module configured to record the on-screen display location of the input response on the display of the patient device and to analyze on-screen location accuracy of the input response, the treatment computer having a communication module configured to transmit the completed treatment data to a bias calculation module, the bias calculation module is configured to to perform treatment session data analysis using the treatment session data selected from display location of stimuli image pairs, input responses, input response latency, treatment session duration, the treatment computer having a reporting module, and the treatment computer operatively connected to secure file storage.

4. A method for calculating an anxiety bias score by measuring latencies between display of stimuli images and patient input, comprising the steps of:
generating, on a patient computer having a computer display screen and input means, an electronic pre-assessment patient profile by receiving a series of patient anxiety profile data inputs into an electronic form displayed on a graphical user interface displayed on the computer display screen, said electronic form having anxiety profile questions relating to feelings selected from fear, apprehension, and worry, and questions relating to physiological symptoms selected from heart palpitations, nausea, chest pain, shortness of breath, and headache;
saving said electronic pre-assessment patient profile to storage memory on a computer at a remote location to the patient computer, said patient computer having computer program code stored in memory and executable on a processor;
providing, on the patient computer, a remotely stored treatment session from a plurality of remotely stored treatment sessions, wherein the remotely stored treatment session is selected by the computer at the remote location operatively connected to the patient computer, said computer at the remote location having computer program code stored in memory and executable on a processor for analyzing the pre-assessment patient profile from the patient computer, and providing the treatment session from the plurality of stored treatment sessions to the patient computer,
wherein the treatment session comprises the steps in order of
(i) displaying patient instructions on the computer display screen,
(ii) displaying a fixation symbol on a central part of the computer display screen for 500 ms or less,
(iii) displaying a set of stimuli images on the computer display screen for a display period of 500 ms or less, said stimuli images selected from negative-neutral stimuli images and neutral-neutral stimuli images, said negative-neutral stimuli images comprising a negative stimuli image displayed at a first location on the computer display screen adjacent to a neutral stimuli image displayed at a second location on the computer display screen, and said neutral-neutral stimuli images comprising a neutral stimuli image displayed at the first location on the computer display screen adjacent to a neutral stimuli image displayed at the second location on the computer display screen,
(iv) displaying a second symbol at the first or second location on the computer display screen for 500 ms or less,
(v) displaying instructions on the computer display screen directing the patient to provide input on the whether the second symbol is displayed at the first or second location,
(vi) receiving patient input on the second symbol location,
(vii) measuring response latencies between displaying the second symbol and receiving patient input, and saving to storage memory response latencies, patient input, location of the second symbol, and type of stimuli images—negative-neutral stimuli images or neutral-neutral stimuli images,
(viii) repeating steps (ii)-(vii) for a length of time selected from a pre-determined length of time, a length of about 20 minutes, and a length of time determined from an analysis of patient responses, wherein the stimuli images from one iteration to the next are different;
displaying the treatment session on the computer display screen of the patient computer, wherein the display period is varied based on the response latencies;
transmitting to the computer at the remote location the response latencies, the patient input, the location of the second symbol, the type of stimuli images—negative-neutral stimuli images or neutral-neutral stimuli images, and the length of time, and said computer at the remote location having computer program code stored in memory and executable on a processor for calculating an anxiety bias score by subtracting response latencies for negative-neutral stimuli images where the location of the second symbol followed the neutral stimuli image (L-neg), from response latencies for neutral-neutral stimuli images (L-neu), and saving the anxiety bias score to storage memory of said computer at the remote location.

5. The method of claim 4, further comprising step (e) performing on said computer at the remote location having computer program code stored in memory and executable on a processor a post-treatment assessment identical to the pre-treatment assessment.

6. The method of claim 4, wherein the patient computer comprises one or more user input devices selected from a mouse, a keyboard, a stylus, touch screen, eye or head movement input, or voice command

7. The method of claim 4, wherein the location of the stimuli images on the computer display screen is varied based on the response latencies.

8. The method of claim 4, wherein the patient profile and the latency data are stored to memory on said computer at the remote location using secure storage technique appropriate to medical information.

9. The method of claim 4, wherein the stimuli images are images of people or words and the anxiety disorder is social anxiety disorder.

10. A process, comprising:
providing a system comprising
a means for displaying one or more sets of stimuli to a patient including a computer having one or more processors, a memory, a screen, and an input means, and
computer storage media comprising computer programming having executable code, wherein the executable code when executed on the one or processors displays the one or more sets of stimuli on a graphical user interface on the screen to the patient and
the computer programming including an interactive computer program that queries the patient to interact with the screen after the one or more sets of stimuli is displayed and subsequently removed, and that is configured to receive a response from the patient on the input means, the one or more sets of stimuli comprising images or words,
displaying a first set of stimuli from the one or more sets of stimuli to the patient on the graphical user interface;
receiving a set of first responses on the input means from the patient to the first set of stimuli;
recording the set of first responses from the patient on the input means to the computer memory and storing the set of first responses for analysis;
displaying a second set of stimuli from the one or more sets of stimuli to the patient, the second set of stimuli prompted by the first set of responses to the first set of stimuli,
receiving a second set of responses on the input means from the patient to the second set of stimuli;
recording the second set of responses from the patient on the input means to the computer memory and storing the second set of responses for analysis;
wherein the one or more sets of stimuli include negative stimuli images or words and neutral stimuli images or words,
wherein each response of the first set of response and the second set of responses is limited to one of two choices given to the patient;
wherein displaying the one or more sets of stimuli comprises first presenting on the display screen a fixation symbol along with an instruction to the patient to focus on the fixation symbol, which subsequently disappears and is replaced by a negative stimuli image or word and a neutral stimuli image or word from the first set of stimuli, the negative stimuli image or word and a neutral stimuli image or word simultaneously displayed around the fixation symbol, along with an instruction to the patient to focus on one of the negative stimuli image or word and a neutral stimuli image or word,
wherein the negative stimuli image or word and a neutral stimuli image or word then simultaneously disappear after a display period of 500 milliseconds or less,
wherein a probe symbol is displayed where one of the negative stimuli image or word and a neutral stimuli image or word was previously positioned on the graphical user interface,
wherein the probe symbol includes an instruction to the patient to enter a response on the input means in a specific manner, the probe symbol remaining on the graphical user interface until the patient enters a response,
wherein recording includes the patient response and a response latency, said response latency comprising the amount of time between the display of the probe symbol and the entry of the response by the patient, each said response is one of the first set of responses or the second set of responses, wherein the display period is varied based on the response latency;
wherein the probe symbol comprises faces selected from a standardized set of emotional expressions,
wherein the standardized set includes faces of eight individuals comprising four male faces and four female faces, the faces of the eight individuals including negative expressions and neutral expressions;
analyzing the first set of responses and the second set of responses that are stored in the memory, by comparing the response latency of each response of the first set of response and the response latency of each response of the second set of responses;
and calculating a bias score by subtracting response latencies where the probe was a negative expression from response latencies where the probe was a neutral expression;
wherein a positive bias score generates a report that the patient has difficulty disengaging attention from a negative stimuli image or word,
wherein a negative bias score generates a report that the patient has reduced anxiety.

11. The process of claim 10, comprising wherein the computer that controls the display of stimuli is local to the patient or is located at a remote location.

12. The process of claim 10, comprising creating a patient profile of the patient by displaying on the graphical user interface a series of questions to assess a level of anxiety in the patient, and obtaining profile answers from the patient using the input means, and storing the patient profile in memory.

13. The process of claim 10, comprising wherein the patient profile automatically selects the instructions and the one or more sets of stimuli.

14. The process of claim 10, comprising wherein the negative stimuli image or word and a neutral stimuli image or word is displayed in the first set of stimuli or the second set of stimuli or both for a time selected from: at least about 50 ms, at least about 50 ms to about 100 ms, at least about 100 ms to about 200 ms, at least about 200 ms to about 300 ms, at least about 300 ms to about 400 ms, at least about 400 ms to about 500 ms, at least about 500 ms to about 600 ms, at least about 600 ms to about 700 ms, at least about 700 ms to about 800 ms, at least about 800 ms to about 900 ms, at least about 900 ms to about 1 second.
